(19) United States
(12) Patent Application Publication
(10) Pub. No.: US 2002/0094981 A1
(43) Pub. Date: Jul. 18, 2002

(54) Ophthalmic compositions for treating ocular hypertension

(75) Inventors: Gerald S. Ponticello, Lansdale, PA (US); Michael F. Sugrue, Blue Bell, PA (US)

Correspondence Address: Merck & Co., Inc., Patent Department, P.O. Box 2000 - RY60-30, Rahway, NJ, 07065-0907, US

(73) Assignee: Merck & Co., Inc.

(21) Appl. No.: 10/023,336

(22) Filed: Dec. 17, 2001

Related U.S. Application Data
(63) Continuation of application No. 09/677,100, filed on Sep. 29, 2000.
Application No. 09/677,100 is a continuation of application No. 09/086,829, filed on May 29, 1998, now abandoned.
(60) Provisional application No. 60/048,140, filed on May 30, 1997.

(51) Int. Cl. A61K 31/5575; A61K 31/542; A61K 31/382

(52) U.S. Cl. 514/226.8; 514/432; 514/530; 514/559

(57) ABSTRACT

Combinations of a prostaglandin or an opthalmologically acceptable salt thereof and a topical carbonic anhydrase inhibitor or an opthalmologically acceptable salt thereof are particularly useful in the treatment of ocular hypertension and glaucoma. The combinations are characterized by an improved effect and reduced side-effects.

This is a continuation of Merck & Co., Inc., attorney docket number 19979 (U.S. Ser. No. 09/086,829) filed May 29, 1998, which was provisionally filed May 30, 1997 as U.S. Ser. No. 60/048,140.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

Many of the drugs formerly used to treat glaucoma proved not entirely satisfactory. The early methods of treatment of glaucoma employing pilocarpine produced undesirable local effects that made this drug, though valuable, unsatisfactory as a first line drug. More recently, clinicians have noted that many β-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use.

Although pilocarpine and β-adrenergic antagonists reduce intraocular pressure, none of these drugs manifests its action by inhibiting the enzyme carbonic anhydrase, and thus they do not take advantage of reducing the contribution to aqueous humor formation made by the carbonic anhydrase pathway.

Agents referred to as carbonic anhydrase decrease the formation of aqueous humor by inhibiting the enzyme carbonic anhydrase. While such carbonic anhydrase inhibitors are now used to treat intraocular pressure by systemic routes, they thereby have the distinct disadvantage of inhibiting carbonic anhydrase throughout the entire body. Such a gross disruption of a basic enzyme system is justified only during an acute attack of alarmingly elevated intraocular pressure, or when no other agent is effective.

For several years, the desirability of directing the carbonic anhydrase inhibitor to only the desired ocular target tissue has been recognized. Because carbonic anhydrase inhibitors have a profound effect in altering basic physiological processes, the avoidance of a systemic route of administation serves to diminish, if not entirely eliminate, those side effects caused by inhibition of carbonic anhydrase such as metabolic acidosis, vomiting, numbness, tingling, general malaise and the like. Topically effective carbonic anhydrase inhibitors are disclosed in U.S. Pat. Nos. 4,386,098; 4,416,890; 4,426,388; 4,668,697; and 4,863,922 and 4,797,413.

Prostaglandins, or Pgs, are members of a class of organic carboxylic acids that are contained in human and most other mammalian tissues or organs and that exhibit a wide range of physiological activities. Naturally occurring Pgs possess a common structural feature, the prostanoic acid skelton, depicted in Formula I below:

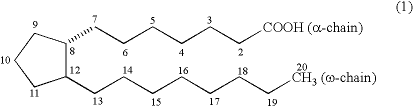

Some synthetic analogues have somewhat modified skeletons. The primary PG's are classified based on the structural feature of the five-membered cycle moiety into PGA's, PGB's, PGC's, PGD's PGE's, PGF's PGG's PGH's PGI's and PGJ's and also on the presence or absence of unsaturation and oxidation in the chain moiety as:

Subscript 1 13,14-unsaturated-15-OH,

Subscript 2 5,6- and 13,14-diunsaturated-15-OH,

Subscript 3 5,6-13,14-, and 17,18-triunsaturated-15-OH

Further, PGFs are subclassified as α or β according to the configuration of the hydroxy group at position 9.

Prostaglandins and prostaglandin derivatives are known to lower intraocular pressure. U.S. Pat. No. 4,883,819 to Bito descibes the use and synthesis of PGAs, PGBs and PGCs in reducing intraocular pressure. U.S. Pat. No. 4,824,857 to Goh et al. describes the use and synthesis of PGD2 and derivatives thereof in lowering intraocular pressure including derivatives wherein C-10 is replaced with nitrogen. U.S. Pat. No. 5,001,153 to Ueno et al. describes the use and synthesis of 13,14-dihydro-15-keto prostaglandins and prostaglandin derivatives to lower intraocular pressure. U.S. Pat. No. 4,599,353 describes the use of eicosanoids and eicosanoid derivatives including prostaglandins and prostaglandin inhibitors in lowering intraocular pressure.

Prostaglandin and prostaglandin derivatives lower intraocular pressure by increasing uveoscleral outflow. This is true for both the F type and A type of Pgs and hence presumably also for the B, C, D, E and J types of prostaglandins and derivatives thereof. A problem with using prostaglandin derivatives to lower intraocular pressure is that these compounds often induce an initial increase in intraocular pressure.

Since the carbonic anhydrase inhibitor lowers intraocular pressure without accompanying transient ocular hypertension exhibited by the primary PGs, the combination of the carbonic anhydrase inhibitor and the prostaglandin derivative can be used for the treatment of diseases and conditions in which the lowering of intraocular pressure is desired, for example glaucoma, ocular hypertension and other disease accompanied by an increase in intraocular pressure.

Thus, when a carbonic anhydrase inhibitor, which decreases the formation of aqueous humor, is combined with a prostaglandin or prostaglandin derivative, which increases the outflow of aqueous humor, there is experienced an effect that reduces intraocular pressure below that obtained by either medicament individually.

The activity of the carbonic anhydrase inhibitor currently marketed wanes 6 to 8 hours post-dose, meaning that as single agents these carbonic anhydrase inhibitors must be administered at least three times day to maintain the desired lowering of intraocular pressure. The combination of this invention maintains the desired lowering of intraocular pessure for a full twelve hours. Because of this increased duration of action, the combination disclosed herein is effective when administered only twice a day. Patient compliance is anticipated to be greater with twice a day administration than with three times a day administration.

The combinations disclosed herein are effective either by co-administration of the medicaments in one solution or as a combined therapy achieved by prior administration of either the carbonic anhydrase inhibitor or the prostaglandin derivative followed by administration of the other solution. The use of a single solution containing both active medicaments is preferred.

There exists a patient population who will benefit from a combination where the minimal dosage of one or both of the medicaments is employed, thus minimizing the possibility of the occurrence of undesirable effects of one or both of the medicaments which would be more likely to become apparent with chronic use at the higher dosage.

### SUMMARY OF THE INVENTION

This invention relates to novel ophthalmic compositions comprising a topical carbonic anhydrase inhibitor or an ophthamologically acceptable salt thereof and a prostaglandin or prostaglandin derivative thereof.

In one aspect of the invention a composition comprising 0.025 to 5% (w/w) of a topical carbonic anhydrase inhibitor such as 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide and their trans and cis enantiomers, or an ophthalmologically acceptable salt thereof, including racemic material and 0.005 to 2% (w/w) of a prostaglandin such as 13,14-dihydro-15(R)-17-phenyl-18,19,20-trinor-PGF2α ester or 13,14-dihydro-15-keto-20-ethyl-PGF2α and their trans and cis enantiomers, or an ophthalmologically acceptable salt thereof, including racemic material, is disclosed. Said composition can optionally contain a gum belonging to the group consisting of gellan gum or xanthan gum.

Another aspect of the invention is concerned with a novel ophthalmic composition comprising comprising 0.025 to 5% (w/w) of a topical carbonic anhydrase inhibitor or an ophthamologically acceptable salt thereof belonging to the group consisting of a compound of structural formula:

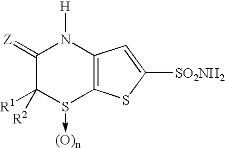

or an ophthalmologically or pharmaceutically acceptable salt thereof, wherein:

Z is (H, H), oxo or thioxo;

R¹ is

(1) hydrogen, or

(2) C₁₋₆ alkyl;

R² is

(1) hydrogen, or

(2) C₁₋₆ alkyl, either unsubstituted or substituted with one or more of

(a) C₁₋₃ alkoxy,

(b) C₁₋₃ alkoxy-(C2-4alkoxy)m-, wherein m is 1-6,

(c) hydroxy,

(d) —NR³R⁴ wherein R³ and R⁴ are independently:

(I) hydrogen

(ii) C₁₋₆ alkyl, either unsubstituted or substituted with one or more of hydroxy, C₁₋₃ alkoxy, C₁₋₃ alkoxy-(C₂₋₄ alkoxy)m-, wherein m is as defined above, or;

(iii) R³ and R⁴taken together with the nitrogen atom to which they are attached represent a saturated heterocycle of 5-7 members which may include a second hetero group selected from N, O, S(O)ₙ, such as piperidine, morpholine, piperazine, N-C1-3 alkylpiperazine, thiomorpholine, thiomorpholine-S-oxide, or thiomorpholineS,S-dioxide;

(e) —CONR³R⁴, where R³ and R⁴ are as defined above,

(f) —CON₃,

(g) —CONHNH₂,

(h) —CO₂H, or

(I) —CO₂R⁵, wherein R⁵ is C₁₋₆ alkyl; and n is 0, 1 or 2, preferably where R1 is hydrogen, Z is (H,H) or oxo, R2 is a Cl-6 substituted alkyl, n is 0 or 2 and and 0.005 to 2% (w/w) of a prostaglandin or prostaglandin derivative thereof. Said composition can be a suspension or a solution.

Another aspect of the invention is concerned with the use of the novel ophthalmic compositions in the treatment of ocular hypertension or glaucoma.

### DETAILED DESCRIPTION OF THE INVENTION

This invention relates to novel ophthalmic combinations comprising a topical carbonic anhydrase inhibitor or an ophthamologically acceptable salt thereof and a prostaglandin or prostaglandin derivative thereof, which are used in the treatment of ocular hypertension and glaucoma.

In one embodiment of this invention, the novel ophthalmic compositions of this invention comprise a pharmaceuticallly acceptable carrier, a therapeutically effective amount of 13,14-dihydro-15(R)-17-phenyl-18,19,20-trinor-PGF2α esters, or 13,14-dihydro-15-keto-20-ethyl-PGF2α isopropryl esters, and a topical carbonic anhydrase inhibitor belonging to the group consisting of 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide and their trans and cis enantiomers, or an ophthalmologically acceptable salt thereof, including racemic material.

An aspect of this invention is realized when the prostaglandin is

11-pivaloyl prostaglandin F2α hydroxyethyl ester,

(+)-(Z)-sodium-7-[1R,2R,3R,5S)-3,5-dihydroxy-2-[(E)-1-octenyl]cyclopentyl]-5-heptenoate sesquihydrate,

[1α,2β,3α,5α]methyl-5-cis-2-(phenylethylsulfonamidomethyl)-3,5-dihydroxycyclopentyl heptenoate,

(+−)-5-[6-(1-hydroxy)hexyl)-1,3-benzodioxol-5-yl]-pentanol, 15-pivaloyl PGFα,

7-[3α,5αdihydroxy-2-(3a-hydroxy-5-1E-pentenyl)cyclopentyl]-5Z-heptenoic acid,

isopropyl (Z)-7-[(1R,2R,3R,5S)-3,5-dihydroxy-2-[(3R)-3-hydroxy-5-phenylpentyl]cyclopentyl]-5-heptenoate or

13,14-dihydro-15-keto-20-ethyl-PGF2α, isopropyl ester trimethylphenol-1-acetate.

A further aspect of this invention is realized when the prostaglandin is isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-l-octenyl]cyclopentyl]-5-heptenoate sesquihydrate, or 13,14-dihydro-15-keto-20-ethyl-PGF2α isopropyl ester trimethylphenol-1-acetate and the topical carbonic anhydrase inhibitor is 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide.

A second embodiment of the invention concerns a composition comprising a topical carbonic anhydrase inhibitor of a compound of structural formula:

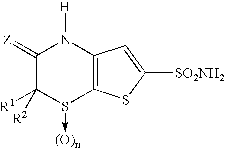

or an ophthalmologically or pharmaceutically acceptable salt thereof, wherein:

Z is (H, H), oxo or thioxo;

R¹ is

(1) hydrogen, or

(2) C₁₋₆ alkyl;

R²is

(1) hydrogen, or

(2) C₁₋₆ alkyl, either unsubstituted or substituted with one or more of

(a) C₁₋₃ alkoxy,

(b) C₁₋₃ alkoxy-(C2-4alkoxy)m-, wherein m is 1-6,

(c) hydroxy,

(d) —NR³R⁴ wherein R³ and R⁴ are independently:

(I) hydrogen

(ii) C₁₋₆ alkyl, either unsubstituted or substituted with one or more of hydroxy, C₁₋₃ alkoxy, C₁₋₃ alkoxy-(C₂₋₄ alkoxy)m-, wherein m is as defined above, or;

(iii) R³ and R⁴ taken together with the nitrogen atom to which they are attached represent a saturated heterocycle of 5-7 members which may include a second hetero group selected from N, 0, S(O)ₙ, such as piperidine, morpholine, piperazine, N-C1-3 alkylpiperazine, thiomorpholine, thiomorpholine—S—oxide, or thiomorpholineS,S-dioxide;

(e) —CONR³R⁴, where R³ and R⁴ are as defined above,

(f) —CON₃,

(g) —CONHNH₂,

(h) —CO₂H, or

(I) —CO₂R⁵, wherein R⁵ is C₁₋₆ alkyl; and n is 0, 1 or 2, preferably where R1 is hydrogen, Z is (H,H) or oxo, R2 is a C1-6 substituted alkyl, n is 0 or 2 and a prostaglandin or prostaglandin derivative.

In one aspect of this invention the topical carbonic anhydrase inhibitor is

2,3-dihydro-2-oxo-6-sulfamoyl-H-thieno-[2,3-b] [1,4]thiazine;

(2,3-dihydro-2-oxo-6-sulfamoyl-H-thieno-[2,3-b] [1,4]thiazin-3-yl)acetic acid;

2,3-dihydro-2,4-dioxo-6-sulfamoyl-H-thieno-[2,3-b][1,4]thiazine;

3-(2-hydroxyethyl)-2,3-dihydro-6-sulfamoyl-1H-thieno[2,3-b] [1,4]thiazine;

3-(2-hydroxyethyl)-2,3-dihydro-4,4-dioxo-6-sulfamoyl-1H-thieno [2,3-b] [1,4]thiazine;

methyl(2,3-dihydro-2,4,4-trioxo-6sulfamoyl-H-thieno-[2,3-b] [1,4]thiazin-3-yl)acetate;

methyl(2,3-dihydro-2-oxo-6-sulfamoyl-H-thieno-[2,3-b] [1,4]thiazin-3-yl)acetate;

N-isobutyl(-2,3-dihydro-2-oxo-6-sulfamoyl-1H-thieno[2,3-b] [1,4]thiazin-3-yl)acetamide;

N-methoxyethoxyethyl-N-methoxyethyl-(2,3-dihydro2,4,4-trioxo-6-sulfamoyl-1H-thieno [2,3-b] [1,4]thiazin-3-yl)acetamide;

3-[2-(N-methoxyethoxyethyl-N-methoxyethyl-amino)ethyl](2,3-dihydro2,4,4-trioxo-6-sulfamoyl-1H-thieno [2,3-b] [1,4]thiazine;

3-(2-isobutylaminoethyl)-2,3-dihydro2,4,4-dioxo-6-sulfamoyl-1H-thieno[2,3-b] [1,4]thiazine;

3-[2-bis-(2-methoxyethyl)aminoethyl]-2,3-dihydro2,4,4-dioxo-6-sulfamoyl-1H-thieno[2,3-b] [1,4]thiazine;

3-[2-(N-methoxyethoxyethyl-N-methoxyethylamino)ethyl]-2,3-dihydro2,4,4-dioxo-6-sulfamoyl-1H-thieno[2,3-b] [1,4]thiazine;

3-(2-morpholinoethyl)-2,3-dihydro2,4,4-dioxo-6-sulfamoyl-1H-thieno[2,3-b] [1,4]thiazine;

and the prostaglandin is

11-pivaloyl prostaglandin F2α hydroxyethyl ester,

(+)-(Z)-sodium-7-[1R,2R,3R,5S)-3,5-dihydroxy-2-[(E)-1-octenyl]cyclopentyl]-5-heptenoate sesquihydrate,

[1α,2β,3α,5α]methyl-5-cis-2-(phenylethylsulfonamidomethyl)-3,5-dihydroxycyclopentyl heptenoate,

(+−)-5-[6-(1-hydroxy)hexyl)-1,3-benzodioxol-5-yl]-pentanol, 15-pivaloyl PGFα,

7-[3α,5α dihydroxy-2-(3a-hydroxy-5-1E-pentenyl)cyclopentyl]-5Z-heptenoic acid,

isopropyl (Z)-7-[(1R,2R,3R,5S)-3,5-dihydroxy-2-[(3R)-3-hydroxy-5-phenylpentyl]cyclopentyl]-5-heptenoate or

13,14-dihydro-15-keto-20-ethyl-PGF2α isopropyl ester trimethylphenol-1-acetate.

A further aspect of this invention is realized when the topical carbonic anhydrase inhibitor is

2,3-dihydro-2-oxo-6-sulfamoyl-H-thieno-[2,3-b] [1,4]thiazine;

(2,3-dihydro-2-oxo-6-sulfamoyl-H-thieno-[2,3-b] [1,4]thiazin-3-yl)acetic acid;

2,3-dihydro-2,4-dioxo-6-sulfamoyl-H-thieno-[2,3-b] [1,4]thiazine;

3-(2-hydroxyethyl)-2,3-dihydro-6-sulfamoyl-1H-thieno[2,3-b][1,4]thiazine;

3-(2-hydroxyethyl)-2,3-dihydro-4,4-dioxo-6-sulfamoyl-1H-thieno [2,3-b] [1,4]thiazine;

methyl(2,3-dihydro-2,4,4-trioxo-6sulfamoyl-H-thieno-[2,3-b] [1,4]thiazin-3-yl)acetate;

methyl(2,3-dihydro-2-oxo-6-sulfamoyl-H-thieno-[2,3-b] [1,4]thiazin-3-yl)acetate;

N-isobutyl(-2,3-dihydro-2-oxo-6-sulfamoyl-1H-thieno[2,3-b] [1,4]thiazin-3-yl)acetamide;

and the prostaglandin is

isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-1-octenyl]cyclopentyl]-5-heptenoate sesquihydrate, or 13,14-dihydro-15-keto-20-ethyl-PGF2α isopropyl ester trimethylphenol-1-acetate.

The term “prostaglandin or prostaglandin derivative”, within this invention refers to those naturally occurring prostaglandins that are useful for lowering intraocular pressure, specifically prostaglandins A, B, C, D, E, F and J class as well as synthetically modified prostaglandins such as 15-keto (oxo group in place of OH at 15) 13,14-dihydro (single bond in place of double bond between positions 13 and 14), and esters thereof. Prostaglandins of the F class, particularly PGF2α derivatives are known to be particularly potent at lowering intraocular pressure.

Although Formula I shows a basic skeleton having twenty carbon atoms, the prostaglandin compounds used in the present invention are not limited to those having the same number of carbon 10 atoms. The carbon atoms in Formula (I) are numbered 2 to 7 on the α-chain starting from the α-carbon atom adjacent to the carboxylic carbon atom which is numbered I and towards the five membered ring 8 to 12 on the ring starting from the carbon atom on which the α-chain is attached, and 13 to 20 on the ω-chain starting from the carbon atom adjacent to the ring. When the number of carbon atoms is decreased on the α-chain, the number is deleted in order starting from position 2 and when the number of carbon atoms is increased in the α-chain compounds are named as substituted derivatives having, substituents at position 1 in place of carboxy group at C-1. Similarly, when the number of carbon atoms is decreased in the ω-chain, the number is deleted in order starting from position 20 and when the number of carbon atoms is increased on the ω-chain, compounds are named as substituted derivatives having respective substituent at position 20. Thus, 13,14-dihydro-15-keto-PG compounds having 10 carbon atoms in the ω-chain are 13,14-dihydro-15-keto-20-ethyl PGs. The term prostaglandin derivative also includes esters of the C-1 carboxyl group, such as the C₁₋₅ alkyl esters.

The novel ophthalmic formulations of this invention comprise about 0.025 to 5% (w/w) of the carbonic anhydrase inhibitors discussed herein, preferably 5,6-dihydro-4-ethylamino-6-methyl-4H-thieno-[2,3-b]thiopyran-2-sulfonamide-7,7 dioxide hydrochloride or 2H-thieno [3,2-e]-1,2-thiazine-6-sulfonamide-4-(ethylamino)-3,4-dihydro-2-(3-methoxypropyl)-1,1-dioxide and their trans and cis enantiomers, or an ophthalmologically acceptable salt thereof, including racemic material, usually about 0.5 to 3% (w/w) and more preferably about 0.7 to about 2% (w/w) and about 0.005 to 2.0% (w/w), preferably about 0.1 to 1% (w/w) of the prostaglandin or prostaglandin derivatives discussed herein, preferablys 13,14-dihydro-15(R)-17-phenyl-18,19,20-trinor-PGF2a esters or 13, 14-dihydro-15-keto-20-ethyl-PGF2α isopropryl esters, and more preferably isopropyl (Z)-7-[(1R,2R,3R,5S)-3,5-dihydroxy-2-[(3R)-3-hydroxy-5-phenylpentyl]cyclopentyl]-5-heptenoate, or 13,14-dihydro-15-keto-20-ethyl-PGF2α isopropyl ester trimethylphenol-1-acetate, to be administered on a 1 to 2 times a day schedule.

A novel method of this invention comprises the topical ocular administration of about 0.025 to about 5 mg per day, preferably about 0.25 to about 3 mg per day of a carbonic anhydrase inhibitor and concomitant, prior, or previous administration of about 0.005 to 2 mg per day, preferably about 0.1 to 1.0 mg per day, of prostaglandin or prostaglandin derivative to each eye.

Suitable subjects for the administration of the formulation of the present invention include mammals, primates, man, and other animals, particularly man and domesticated animals such as cats and dogs. For topical ocular administration the novel formulations of this invention may take the form of solutions, gels, ointments, suspensions or solid inserts, formulated so that a unit dosage comprises a therapeutically effective amount of each active component or some submultiple thereof.

Typical ophthalmologically acceptable carriers for the novel formulations are, for example, water, mixtures of water and water-miscible solvents such as lower alkanols or aralkanols, vegetable oils, polyalkylene glycols, petroleum based jelly, ethyl cellulose, ethyl oleate, carboxymethylcellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, benzalkonium chloride, methyl and propyl paraben, benzyldodecinium bromide, benzyl alcohol, phenylethanol, buffering ingredients such as sodium chloride, sodium borate, sodium acetate, or gluconate buffers, and other conventional ingredients such as sorbitan monolaurate, triethanolamine, polyoxyethylene sorbitan monopalmitylate, dioctyl sodium sulfosuccinate, monothioglycerol, thiosorbitol, ethylenediamine tetra acetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehhicles and the like.

The formulation may also include a gum such as gellan gum at a concentration of 0. 1% to 2% by weight so that the aqueous eyedrops gel on contact with the eye, thus providing the advantages of a solid ophthalmic insert as described in U.S. Pat. No. 4,861,760.

The formulation may also include a gum such as xanthan gum at a concentration of 0.1 to 2%, preferably 0.4 to 0.7%(w/w). Particularly preferred is KELTROL T xanthan gum from Monsanto Performance Materials. The formulation of the instant invention employing xanthan gum will be a hypotonic solution, with a freezing point depression between about −0.28 C. and −0.4 C., and preferably between about −0.31 C. and −0.37 C. Alternatively, the hypotonicity of the ophthalmic solutoins of the present invention employing xanthan gum will be between about 150 and 215 mOs/kg, and preferably between 170 and 200 mOs/kg. Coventional ophthalmic solutions are usually prepared as isotonic solutions using tonicity adjusting agents as potassium chloride, sodium chloride, mannitol, dextrose and glycerin. An isotonic solution will have a freezing point depression of approximately −0.54 C. Tonicity may also be measured by the osmolality of the solution, an isotonic solution having an osmolality of about 290 milliosmoles per kilogram (mOs/kg).

The pharmaceutical preparation may also be in the form of a solid insert such as one which after dispensing the drug remains essentially intact as described in U.S. Pat. No. 4,256,108; 4,160,452; and 4,265,874; or a bio-erodible insert that either is soluble in lacrimal fluids, or otherwise disintegrates as described in U.S. Pat. No. 4,287,175 or EPO publication 0,077,261.

The pharmaceutical preparation may also be in the form of a suspension utilizing carbonic anhydrase inhibitors (CAI's) having aqueous solubilities greater than 10 g/mL but less than 1000 μg/mL at pH 7.4, octanol/water distribution coefficients (DC) measured at pH 7.4 of from 1.0 to 150 and dissociation constants (Kᵢ) of 1.0 nM or lower. The aqueous solubility is measured, for example, by mixing the CAI, in its neutral or salt form in 0.1M phosphate buffer at a pH of 7.4. The mixture is then agitated for approximately 16 to 24 hours, while maintaining a pH of 7.4. If the mixture is a solution, a small amount of a seed crystal of the neutral CAI is added and the mixture is stirred for approximately 16 to 24 hours. The solid/liquid mixture is filtered throught a 0.45 μm filter and the filtrated is assayed by HPLC against standards. The solubility as measured includes both the neutral and ionized forms of the CAI. Under these conditions, at pH 7.4, the CAI's employed for the suspension are predominantly unionized, with the possibility of 10 to 20% of the anionic sulfonamide present (depending on the pKa of the primary sulfonamide group). By way of an example, the suspension encompassed within the meaning of this invention is one which comprises 0.1-10.9 wt % of a carbonic anhydrase inhibitor and 0.01-10.0 wt. % of a polyethoxylated derivative of castor oil resulting from the reaction of from 2-200 moles of ethylene oxide per 1 mole of castor oil, wherein the derivatives can be hydrogenated.

The measure of the dissociation constant is determined using the fluorescence competition assay which uses the fluorescent HCAII:dansylamide complex and is well known in the art, Chen et al., J. Biol. Chem., 242, 5813 (1967) and Ponticello et al., J. Med. Chem., 30, 591 (1987). The relative Kis for the suspension are less than 3.3.

The following examples of ophthalmic formulations are given by way of illustration and are not limitative of the invention.

EXAMPLE 1

| SOLUTION COMPOSITION | I | | ii | | III | |
|---|---|---|---|---|---|---|
| (S,S)-(-)-5,6-dihydro-4-ethyl- | 22.26 | g | 22.26 | g | 1.113 | g |
| amino-6-methyl-4H-thieno- | | | | | | |
| [2,3b]thiopyran-2-sulfonamide- | | | | | | |
| 7,7-dioxide monohydrochloride | | | | | | |
| (carbonic anhydrase inhibitor) | | | | | | |
| 13,14-dihydro-15-keto-20-ethyl- | 10.0 | g | 1.0 | g | 1.0 | g |
| PGF2. isopropyl ester | | | | | | |
| (prostaglandin derivative) | | | | | | |
| Sodium citrate.2H2O | 2.940 | g | 2.940 | g | 2.940 | g |
| Benzalkonium Chloride | 0.075 | g | 0.075 | g | 0.075 | |
| Hydroxyethylcellulose | 5.00 | g | 5.00 | g | 5.00 | g |
| Sodium hydroxide q.s. | pH = 6.0 | | pH = 6.0 | | pH = 6.0 | |
| Mannitol | 16.00 | g | 21.00 | g | 35.90 | g |
| Water for injection q.s. ad. | 1000 | g | 1000 | g | 1000 | g |

The active compounds, phosphate buffer salts, benzalkonium chloride, and Polysorbate 80 are added to and suspended or dissolved in water. The pH of the composition is adjusted to 5.5-6.0 and diluted 30 to volume. The composition is rendered sterile by filtration through a sterilizing filter.

EXAMPLES 2-6
Following the procedures of Example 1, solutions are
prepared substituting the compounds below for the
carbonic anhydrase inhibitors:

| Compound | Example No. |
|---|---|
| (S,S)-(-)-5,6-dihydro-4-ethylamino- | 2 |
| 6-methyl-4H-thieno[2,3b]thiopyran- | |
| 2-sulfonamide-7,7-dioxide | |
| (S,S)-(-)-3,4-dihydro-4-ethylamino-2-methyl- | 3 |
| 2H-thieno[3,2-e]-1,2-thiazine-6-sulfon- | |
| amide-1,1-dioxide hydrochloride | |
| R-(+)-3,4-dihydro-4-ethylamino-2- | 4 |
| methyl-2H-thieno[3,2-e]-1,2- | |
| thiazine-6-sulfonamide-1,1-dioxide | |
| hydrochloride | |
| R-(+)-3,4-dihydro-4-ethylamino-2- | 5 |
| (2-methoxy)ethyl-2H-thieno[3,2-e]-1,2- | |
| thiazine-6-sulfonamide-1,1-dioxide | |
| hydrochloride | |
| (S,S)-(-)-5,6-dihydro-4-ethylamino- | 6 |
| 6-propyl-4H-thieno[2,3b]thiopyran- | |
| 2-sulfonamide-7,7-dioxide | |

EXAMPLE 7

| | CONCENTRATION |
|---|---|
| Suspension | (WT/V%) |
| R-(+)-4-ethylamino-3,4-dihydro-2-(3-methoxy) | 2% + 2% xs |
| propyl-2H-thieno[3,2-e]-1,2-thiazine-6-sulfon- | |
| amide 1,1-dioxide | |
| 13,14-dihydro-15-keto-20-ethyl- | 0.5% |
| PGF2. isopropyl ester | |
| (prostaglandin derivative) | |
| Hydroxypropylmethylcellulose | 3% |
| Dibasic Sodium Phosphate | 0.2% |
| Sodium Chloride | 0.7% |
| Disodium Edetate | 0.01% |
| Polysorbate 80 | 0.05% |
| Benzalkonium Chloride | 0.01% |
| NaOH/HC1 | pH adjust |
| Purified Water | q.s. 100% |

The suspension may be prepared by heating 400 mL of purified water to boiling. HPMC (30.0 g) is added and the mixture stirred vigorously until homegeneous. To this is added a solution consisting of sodium chloride (7.0 g), dibasic sodium phosphate (2.0 g), disodium edta (0.1 g), polysorbate 80 (0.5 g) and benzalkonium chloride (10.5 mL of a 1% solution) and purified water is added to a final volume of 900 mL. The mixture is stirred and cooled in an ice bath to room temperature and the pH is adjusted to 7.2 employing HCl (3.5 mL of a 1 N solution. The mixture is q.s. to the final weight with purified water (total 1010 g) and filtered through a 10 micron filter. The formulation is prepared by the addition of the above HPMC vehicle (15.014 g) to the above TCAI (0.3074 g) and prostaglandin (1.0 g) and the mixture ias ball milled with 3 mm glass beads (5 g) for approximately 45 hours.

### EXAMPLES 8-12

Following the procedures of Example 1, solutions are prepared substituting the compounds below for the prostaglandin derivative

EXAMPLES 8-12
Following the procedures of Example 1, solutions are
prepared substituting the compounds below for the
prostaglandin derivative

| Compound | Example No. |
|---|---|
| PGF2α,-1-isopropyl ester | 8 |
| PGA2 | 9 |
| 13,14-dihydro-15-keto-PGE2 | 10 |
| methyl ester | |
| 15-keto-PGF2α | 10 |
| PGF2α tromethamine salt | 11 |
| PGA1 | 12 |

EXAMPLE 13

| SOLUTION COMPOSITION | I | | II | |
|---|---|---|---|---|
| 5,6-dihydro-4-ethylamino | 2.0 | mg | 0.2 | mg |
| 6-methyl-4H-thieno[2,3b]thiopyran- | | | | |
| 2-sulfonamide-7,7-dioxide | | | | |
| monohydrochloride | | | | |
| (carbonic anhydrase inhibitor) | | | | |
| 13,14-dihydro-15-keto-20-ethyl- | 0.1 | mg | 1.0 | mg |
| PGF2α isopropyl ester | | | | |
| trimethylphenol-1-acetate | | | | |
| Gelrite™ gellan gum | 6.0 | mg | 6.0 | mg |
| Monobasic sodium phosphate | Quantity sufficient to | | | |
| | give .2H20 | | | |
| Dibasic sodium phosphate | final pH | | 5.5-6.0 | |
| .12H20 | | | | |
| Benzyldodecinium bromide | 0.10 | mg | 0.10 | mg |
| Polysorbate 80 | 0.2 | mg | 0.2 | mg |
| Water for injection q.s. ad. | 1.0 | mL | 1.0 | mL |

The active compounds, Gelrite' gellan gum, phosphate buffer salts, benzyldodecinium bromide and Polysorbate 80 are added to and suspended or dissolved in water. The pH of the composition is adjusted to 5.5-6.0 and diluted to volume. The composition is rendered sterile by ionizing radiation.

EXAMPLES 14-18
Following the procedures of Example 13, solutions are prepared
substituting the compounds below for the carbonic
anhydrase inhibitors:

| Compound | Example No. |
|---|---|
| (S,S)-(-)-5,6-dihydro-4-ethylamino- | 14 |
| 6-methyl-4H-thien6[2,3b]thiopyran- | |
| 2-sulfonamide-7,7-dioxide | |
| 3,4-dihydro-4-ethylamino-2-methyl- | 15 |
| 2H-thieno[3,2-e]-1,2-thiazine-6-sulfon- | |
| amide-1,1 dioxide hydrochloride | |
| R-(+)-3,4-dihydro-4-ethylamino-2- | 16 |
| methyl-2H-thieno[3,2-e]-1,2- | |
| thiazine-6-sulfonamide-1,1-dioxide | |
| hydrochloride | |
| R-(+)-3,4-dihydro-4-ethylamino-2- | 17 |
| (2-methoxy)ethyl-2H-thieno[3,2-e]-1,2- | |
| thiazine-6-sulfonamide-1,1-dioxide | |
| hydrochloride | |
| (S,S)-trans-5,6-dihidro-4-ethylamino- | 18 |
| 6-propyl-4H-thieno[2,3b]thiopyran- | |
| 2-sulfonamide-7,7-dioxide | |

EXAMPLES 19-24
Following the procedures of Example 13, solutions are
prepared substituting the compounds below for the
prostaglandin derivative.

| Compound | Example |
|---|---|
| PGF2(x-1-isopropyl ester | 19 |
| PGA2 | 20 |
| 13,14-dihydro-15-keto-PGE2 | 21 |
| methyl ester | |
| 15-keto-PGF,2α | 22 |
| PGF2α tromethamine salt | 23 |
| PGA₁ | 24 |

EXAMPLE 25

| SOLUTION COMPOSITION | I | II |
|---|---|---|
| 5,6-dihydro-4-ethylamino | 2% | 2% |
| 6-methyl-4H-thieno[2,3b]thiopyran- | | |
| 2-sulfonamide-7,7-dioxide | | |
| monohydrochloride | | |
| (carbonic anhydrase inhibitor) | | |
| 13,14-dihydro-15-keto-20-ethyl- | 0.1% | 1.0% |
| PGF2α isopropyl ester | | |
| trimethylphenol-1-acetate | | |
| Xanthan gum | 0.5% | 0.7% |
| Sodium Chloride | 0.2% | 0.2% |
| Benzalkonium Chloride | 0.0075% | 0.0075% |
| Sodium Hydroxide | qs pH5.6 | pH 5.6 |
| Water | qs 100% | 100% |

The active compounds, sodium chloride and benzalkonium chloride are dissolved in water for injection. The pH of the composition is adjusted to 5.6 by addition of 0.2N sodium hydroxide solution, and water for injection is added until the weight of the composition is equal to 75 parts of the final weight (I) or 65 parts of the final weight (II). The composition is sterilized by filtration, and the solution flushed with sterile nitrogen. Then a clarified, steam sterilized concentrate of 2% xanthan gum is added to the solution of drug and the resulting solution is homogenized by stirring. The solution is aseptically subdivided into sterile vials and sealed.

EXAMPLES 26-30
Following the procedures of Example 13, solutions are pre-
pared substituting the compounds below for the carbonic
anhydrase inhibitors:

| Compound | Example No. |
|---|---|
| (S,S)-(-)-5,6-dihydro-4-ethylamino- | 26 |
| 6-methyl-4H-thien6[2,3b]thiopyran- | |
| 2-sulfonamide-7,7-dioxide | |
| 3,4-dihydro-4-ethylamino-2-methyl- | 27 |
| 2H-thieno[3,2-e]-1,2-thiazine-6-sulfon- | |
| amide-1,1 dioxide hydrochloride | |
| R-(+)-3,4-dihydro-4-ethylamino-2- | 28 |
| methyl-2H-thieno[3,2-e]-1,2- | |
| thiazine-6-sulfonamide-1,1-dioxide | |
| hydrochloride | |
| R-(+)-3,4-dihydro-4-ethylamino-2- | 29 |
| (2-methoxy)ethyl-2H-thieno[3,2-e]-1,2- | |
| thiazine-6-sulfonamide-1,1-dioxide | |
| hydrochloride | |
| (S,S)-trans-5,6-dihidro-4-ethylamino- | 30 |
| 6-propyl-4H-thieno[2,3b]thiopyran- | |
| 2-sulfonamide-7,7-dioxide | |

EXAMPLES 31-36
Following the procedures of Example 13, solutions are
prepared substituting the compounds below for the
prostaglandin derivative.

| Compound | Example |
|---|---|
| PGF2α-1-isopropyl ester | 31 |
| PGA2 | 32 |
| 13,14-dihydro-15-keto-PGE2 | 33 |
| methyl ester | |
| 15-keto-PGF,2α | 34 |
| PGF2α tromethamine salt | 35 |
| PGA₁ | 36 |

What is claimed is:

1. An ophthalmic formulation for the treatment of ocular hypertension and glaucoma in a subject in need thereof, comprising an ophthalmologically acceptable carrier, 0.025 to 5% (w/w) of a carbonic anhydrase inhibitor belonging to the group consisting of 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide and 0.005 to 2% (w/w) of a prostaglandin belonging to the group consisting of 13,14-dihydro-15(R)-17-phenyl-18,19,20-trinor-PGF2α esters, or 13,14-dihydro-15-keto-20-ethyl-PGF2α isopropryl esters, and their trans and cis enantiomers, or an ophthalmologically acceptable salt thereof, including racemic material.

2. A formulation according to claim 1 wherein the prostaglandin is
11-pivaloyl prostaglandin F2α hydroxyethyl ester,
(+)-(Z)-sodium-7-[1R,2R,3R,5S)-3,5-dihydroxy-2-[(E)-1-octenyl]cyclopentyl]-5-heptenoate sesquihydrate,
[1α,2β,3α,5α]methyl-5-cis-2-(phenylethylsulfonamidomethyl)-3,5-dihydroxycyclopentyl heptenoate,
(+−)-5-[6-(1-hydroxy)hexyl)-1,3-benzodioxol-5-yl]-pentanol, 15-pivaloyl PGFα,
7-[3α,5αdihydroxy-2-(3a-hydroxy-5-1E-pentenyl)cyclopentyl]-5Z-heptenoic acid,
isopropyl (Z)-7-[(1R,2R,3R,5S)-3,5-dihydroxy-2-[(3R)-3-hydroxy-5-phenylpentyl]cyclopentyl]-5-heptenoate or
13,14-dihydro-15-keto-20-ethyl-PGF2α isopropyl ester trimethylphenol-1-acetate.

3. A formulation according to claim 1 wherein the prostaglandin is isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-1-octenyl]cyclopentyl]-5-heptenoate sesquihydrate, or 13,14-dihydro-15-keto-20-ethyl-PGF2cc isopropyl ester trimethylphenol-1-acetate and the topical carbonic anhydrase inhibitor is 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide.

4. A formulation according to claim 3 wherein the prostaglandin is isopropyl (Z)-7-[(1R,2R,3R,5S)-3,5-dihydroxy-2-[(3R)-3-hydroxy-5-phenylpentyl]cyclopentyl]-5-heptenoate and the topical carbonic anhydrase inhibitor is 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide.

5. A formulation according to claim 3 wherein the prostaglandin is 13,14-dihydro-15-keto-20-ethyl-PGF2α isopropyl ester trimethylphenol-1-acetate and the topical carbonic anhydrase inhibitor is 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide.

6. A formulation according to claim 3 wherein the prostaglandin is (+)-(Z)-sodium-7-[1R,2R,3R,5S)-3,5-dihydroxy-2-[(E)-1-octenyl]cyclopentyl]-5-heptenoate sesquihydrate and the topical carbonic anhydrase inhibitor is 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide.

7. An ophthalmic formulation for the treatment of ocular hypertension and glaucoma in a subject in need thereof, comprising an ophthalmologically acceptable carrier, 0.025 to 5% (w/w) of a carbonic anhydrase inhibitor belonging to the group consisting of a compound of structural formula: 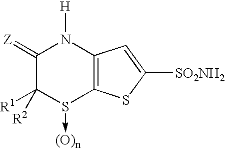
or an ophthalmologically or pharmaceutically acceptable salt thereof, wherein:
Z is (H, H), oxo or thioxo;
R¹ is
(1) hydrogen, or
(2) C₁₆ alkyl;
R² is
(1) hydrogen, or
(2) C₁₋₆ alkyl, either unsubstituted or substituted with one or more of
(a) C₁₋₃ alkoxy,
(b) C₁₋₃ alkoxy-(C2-4alkoxy)m-, wherein m is 1-6,
(c) hydroxy,
(d) —NR³R⁴ wherein R³ and R⁴ are independently:
(I) hydrogen
(ii) C₁₋₆ alkyl, either unsubstituted or substituted with one or more of hydroxy, C₁₋₃ alkoxy, C₁₋₃ alkoxy-(C₂₋₄ alkoxy)m-, wherein m is as defined above, or;
(iii) R³ and R⁴ taken together with the nitrogen atom to which they are attached represent a saturated heterocycle of 5-7 members which may include a second hetero group selected from N, 0, S(O)ₙ, such as piperidine, morpholine, piperazine, N-C1-3 alkylpiperazine, thiomorpholine, thomorpholine—S—oxide, or thiomorpholineS ,S -dioxide;
(e) —CONR³R⁴, where R³ and R⁴ are as defined above,
(f) —CON₃,
(g) —CONHNH₂,
(h) —CO₂H, or
(I) —CO₂R⁵, wherein R⁵ is C₁₆ alkyl; and n is 0, 1 or 2, and 0.005 to 2% (w/w) of a prostaglandin or prostaglandin derivative or an opthalmologically acceptable salt thereof.

8. The formulation of claim 7 wherein R¹ is hydrogen, Z is (H,H) or oxo, R² is a C₁₋₆ substituted alkyl and n is 0 or 2.

9. The formulation of claim 7 wherein the topical carbonic anhydrase inhibitor belongs to the group consisting of
2,3-dihydro-2-oxo-6-sulfamoyl-H-thieno-[2,3-b] [1,4]thiazine;
(2,3-dihydro-2-oxo-6-sulfamoyl-H-thieno-[2,3-b] [1,4]thiazin-3-yl)acetic acid;
3-dihydro-2,4-dioxo-6-sulfamoyl-H-thieno-[2,3-b] [1,4]thiazine;
3-(2-hydroxyethyl)-2,3-dihydro-6-sulfamoyl-1H-thieno[2,3-b] [1,4]thiazine;
3-(2-hydroxyethyl)-2,3-dihydro-4,4-dioxo-6-sulfamoyl-1H-thieno[2,3-b] [1,4]thiazine;
methyl(2,3-dihydro-2,4,4-trioxo-6sulfamoyl-H-thieno-[2,3-b] [1,4]thiazin-3-yl)acetate;
methyl(2,3-dihydro-2-oxo-6-sulfamoyl-H-thieno-[2,3-b] [1,4]thiazin-3-yl)acetate;
N-isobutyl(-2,3-dihydro-2-oxo-6-sulfamoyl-1H-thieno[2,3-b] [1,4]thiazin-3-yl)acetamide;
N-methoxyethoxyethyl-N-methoxyethyl-(2,3-dihydro2,4,4-trioxo-6-sulfamoyl-1H-thieno [2,3-b] [1,4]thiazin-3-yl)acetamide;
3-[2-(N-methoxyethoxyethyl-N-methoxyethyl-amino)ethyl] (2,3-dihydro2,4,4-trioxo-6-sulfamoyl-1H-thieno[2,3-b] [1,4]thiazine;
3-(2-isobutylaminoethyl)-2,3-dihydro2,4,4-dioxo-6-sulfamoyl-1H-thieno[2,3-b][1,4]thiazine;
3-[2-bis-(2-methoxyethyl)aminoethyl]-2,3-dihydro2,4,4-dioxo-6-sulfamoyl-1H-thieno [2,3-b] [1,4]thiazine;
3-[2-(N-methoxyethoxyethyl-N-methoxyethylamino)ethyl]-2,3-dihydro2,4,4-dioxo-6-sulfamoyl-1H-thieno[2,3-b] [1,4]thiazine;
3-(2-morpholinoethyl)-2,3-dihydro2,4,4-dioxo-6-sulfamoyl-1H-thieno[2,3-b][1,4]thiazine;
and the prostaglandin is
11-pivaloyl prostaglandin F2α hydroxyethyl ester,
(+)-(Z)-sodium-7-[1R,2R,3R,5S)-3,5-dihydroxy-2-[(E)-1-octenyl]cyclopentyl]-5-heptenoate sesquihydrate
[1α,2β,3α,5α]methyl-5-cis-2-(phenylethylsulfonamidomethyl)-3,5-dihydroxycyclopentyl heptenoate,
(+−)-5-[6-(1-hydroxy)hexyl)-1,3-benzodioxol-5-yl]-pentanol, 15-pivaloyl PGFα,
7-[3α,5αdihydroxy-2-(3a-hydroxy-5-1E-pentenyl)cyclopentyl]-5Z-heptenoic acid,
isopropyl (Z)-7-[(1R,2R,3R,5S)-3,5-dihydroxy-2-[(3R)-3-hydroxy-5-phenylpentyl]cyclopentyl]-5-heptenoate or
13,14-dihydro-15-keto-20-ethyl-PGF2α isopropyl ester trimethylphenol-1-acetate.

10. A formulation according to claim 9 wherein the topical carbonic anhydrase inhibitor is
2,3-dihydro-2-oxo-6-sulfamoyl-H-thieno-[2,3-b] [1,4]thiazine;
(2,3-dihydro-2-oxo-6-sulfamoyl-H-thieno-[2,3-b] [1,4]thiazin-3-yl)acetic acid;
2,3-dihydro-2,4-dioxo-6-sulfamoyl-H-thieno-[2,3-b] [1,4]thiazine;
3-(2-hydroxyethyl)-2,3-dihydro-6-sulfamoyl-1H-thieno[2,3-b] [1,4]thiazine;
3-(2-hydroxyethyl)-2,3-dihydro-4,4-dioxo-6-sulfamoyl-1H-thieno[2,3-b][1,4]thiazine;
methyl(2,3-dihydro-2,4,4-trioxo-6sulfamoyl-H-thieno-[2,3-b] [1,4]thiazin-3-yl)acetate;
methyl(2,3-dihydro-2-oxo-6-sulfamoyl-H-thieno-[2,3-b] [1,4]thiazin-3-yl)acetate;
N-isobutyl(-2,3-dihydro-2-oxo-6-sulfamoyl-1H-thieno[2,3-b] [1,4]thiazin-3-yl)acetamide;
and the prostaglandin is
isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,
5S)-3,5-dihydroxy-2-[(E)-1-octenyl]cyclopentyl]-5-heptenoate sesquihydrate or
13,14-dihydro-15-keto-20-ethyl-PGF2α isopropyl ester trimethylphenol-1-acetate.

11. The formulation of claim 1 wherein the concentration of carbonic anhydrase inhibitor is 0.5% to 3% and the concentration of the prostaglandin or prostaglandin derivative is 0.1% to 1.0%.

12. The formulation of claim 7 wherein the concentration of carbonic anhydrase inhibitor is 0.5% to 3% and the concentration of the prostaglandin or prostaglandin derivative is 0.1% to 1.0%.

13. The formulation of claim 12 wherein the carbonic anhydrase inhibitor has an aqueous solubility greater than 10 ug/mL but less than 1000 ug/mL at pH 7.4, and a Ki of 1.0 nM or lower.

14. The formulation of claim 13 which is a suspension.

15. The formulation of claim 1 which optionally contains from about 0. 1 % to about 2% of gellan gum.

16. The formulation of claim 1 which optionally contains from about 0.1 % to about 2% (w/w) of xanthan gum.

17. The formulation of claim 16 which contains from about 0.4 to about 0.7%(w/w) of xanthan gum, said xanthan gum being a hypotonic solution, with a freezing point depression between about −0.28 C. and −0.4 C.

18. The formulation of claim 17 wherein the gum is KELTROL T xanthan gum in a hypotonic solution with a freezing point from about −0.31 C. to about -0.37 C.

19. A method of treating ocular hypertension and glaucoma which comprises the topical ocular administration to a patient in need of such treatment of a unit dose of the formulation of claim 1.

20. A method of treating ocular hypertension and glaucoma which comprises the topical ocular administration to a patient in need of such treatment of a unit dose of the formulation of claim 7.

21. A method of treating ocular hypertension and glaucoma which comprises the topical ocular administration to a patient in need of such treatment of a unit dose of the formulation of claim 14.

22. A method of treating ocular hypertension and glaucoma which comprises the topical ocular administration to a patient in need of such treatment of a unit dose of the formulation of claim 15.

23. A method of treating ocular hypertension and glaucoma which comprises the topical ocular administration to a patient in need of such treatment of a unit dose of the formulation of claim 16.

24. A method of treating ocular hypertension and glaucoma which comprises the topical ocular administration to a patient in need of such treatment of a unit dose of the formulation of claim 17.

25. A method of treating ocular hypertension and glaucoma which comprises the topical ocular administration to a patient in need of such treatment of a unit dose of the formulation of claim 8.

26. An ophthalmic formulation for the treatment of ocular hypertension and glaucoma in a subject in need thereof, comprising an ophthalmologically acceptable carrier, 0.5 to 3% (w/w) of a carbonic anhydrase inhibitor belonging to the group consisting of 5,6-dihydro-4-ethylamino-6-methyl-4H-thieno-[2,3-b]thiopyran-2-sulfonamide-7,7 dioxide hydrochloride or 2H-thieno[3,2-e]-1,2-thiazine-6-sulfonamide-4-(ethylamino)-3,4-dihydro-2-(3-methoxypropyl)-1,1-dioxide 0.1% to 1.0% (w/w) of a prostaglandin belonging to the group consisting of 11-pivaloyl prostaglandin F2α hydroxyethyl ester,
(+)-(Z)-sodium-7-[1R,2R,3R,5S)-3,5-dihydroxy-2-[(E)-1-octenyl]cyclopentyl]-5-heptenoate sesquihydrate,
[1α,2β,3α,5α]methyl-5-cis-2-(phenylethylsulfonamidomethyl)-3,5-dihydroxycyclopentyl heptenoate,
(+−)-5-[6-(1-hydroxy)hexyl)-1,3-benzodioxol-5-yl]-pentanol, 15-pivaloyl PGFα,
7-[3α,5α dihydroxy-2-(3a-hydroxy-5-1E-pentenyl)cyclopentyl]-5Z-heptenoic acid,
isopropyl (Z)-7-[(1R,2R,3R,5S)-3,5-dihydroxy-2-[(3R)-3-hydroxy-5-phenylpentyl]cyclopentyl]-5-heptenoate or
13,14-dihydro-15-keto-20-ethyl-PGF2α isopropyl ester trimethylphenol-1-acetate, and their trans and cis enantiomers, or an ophthalmologically acceptable salt thereof, including racemic material, and a gum belonging to the group consisting of from about 0.1% to about 2% of gellan gum or from about 0.1 % to about 2% (w/w) of xanthan gum.

27. A formulation according to claim 26 wherein the carbonic anhydrase inhibitor is 5,6-dihydro-4-ethylamino-6-methyl-4H-thieno-[2,3-b]thiopyran-2-sulfonamide-7,7 dioxide hydrochloride, the prostaglandin is isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-1-octenyl]cyclopentyl]-5-heptenoate sesquihydrate or
13,14-dihydro-15-keto-20-ethyl-PGF2α isopropyl ester trimethylphenol-1-acetate and the gum is gellan gum.

28. A formulation according to claim 26 wherein the carbonic anhydrase inhibitor is 5,6-dihydro-4-ethylamino-6-methyl-4H-thieno-[2,3-b]thiopyran-2-sulfonamide-7,7 dioxide hydrochloride, the prostaglandin is isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-l-octenyl]cyclopentyl]-5-heptenoate sesquihydrate or 13,14-dihydro-15-keto-20-ethyl-PGF2α isopropyl ester trimethylphenol-1-acetate and the gum is xanthan gum.

29. A formulation according to claim 28 which contains from about 0.4 to about 0.7%(w/w) of xanthan gum, said xanthan gum being a hypotonic solution, with a freezing point depression between about −0.28 C. and −0.4 C.

30. The formulation of claim 29 wherein the gum is KELTROL T xanthan gum in a hypotonic solution with a freezing point from about −0.31 C. to about −0.37 C.

31. An ophthalmic formulation for the treatment of ocular hypertension and glaucoma in a subject in need thereof, comprising an ophthalmologically acceptable carrier, 0.5 to 3% (w/w) of a carbonic anhydrase inhibitor belonging to the group consisting of
2,3-dihydro-2-oxo-6-sulfamoyl-H-thieno-[2,3-b] [1,4]thiazine;
(2,3-dihydro-2-oxo-6-sulfamoyl-H-thieno-[2,3-b] [ 1,4]thiazin-3-yl)acetic acid;
2,3-dihydro-2,4-dioxo-6-sulfamoyl-H-thieno-[2,3-b] [1,4]thiazine;
3-(2-hydroxyethyl)-2,3-dihydro-6-sulfamoyl-1H-thieno[2,3-b] [1,4]thiazine;
3-(2-hydroxyethyl)-2,3-dihydro-4,4-dioxo-6-sulfamoyl-1H-thieno-[2,3-b] [1,4]thiazine;
methyl(2,3-dihydro-2,4,4-trioxo-6sulfamoyl-H-thieno-[2,3-b] [1,4]thiazin-3-yl)acetate;
methyl(2,3-dihydro-2-oxo-6-sulfamoyl-H-thieno-[2,3-b][1,4]thiazin-3-yl)acetate;
N-isobutyl(-2,3-dihydro-2-oxo-6-sulfamoyl-1H-thieno [2,3-b] [1,4]thiazin-3-yl)acetamide;
N-methoxyethoxyethyl-N-methoxyethyl-(2,3-dihydro2,4,4-trioxo-6-sulfamoyl-1H-thieno[2,3-b] [1,4]thiazin-3-yl)acetamide;
3-[2-(N-methoxyethoxyethyl-N-methoxyethyl-amino)ethyl](2,3-dihydro2,4,4-trioxo-6-sulfamoyl-1H-thieno[2,3-b] [1,4]thiazine;
3-(2-isobutylaminoethyl)-2,3-dihydro2,4,4-dioxo-6-sulfamoyl-1H-thieno [2,3-b] [1,4]thiazine;
3-[2-bis-(2-methoxyethyl)aminoethyl]-2,3-dihydro2,4,4-dioxo-6-sulfamoyl-1H-thieno[2,3-b] [1,4]thiazine;
3-[2-(N-methoxyethoxyethyl-N-methoxyethylamino)ethyl]-2,3-dihydro2,4,4-dioxo-6-sulfamoyl-1H-thieno[2,3-b] [1,4]thiazine;
3-(2-morpholinoethyl)-2,3-dihydro2,4,4-dioxo-6-sulfamoyl-1H-thieno[2,3-b][1,4]thiazine;
and about 0. % to about 1% of a prostaglandin consisting of 11-pivaloyl prostaglandin F2α hydroxyethyl ester,
(+)-(Z)-sodium-7-[1R,2R,3R,5S)-3,5-dihydroxy-2-[(E)-1-octenyl]cyclopentyl]-5-heptenoate sesquihydrate,
[1α,2β,3α,5α]methyl-5-cis-2-(phenylethylsulfonamidomethyl)-3,5-dihydroxycyclopentyl heptenoate,
(+−)-5-[6-(1-hydroxy)hexyl)-1,3-benzodioxol-5-yl]-pentanol, 15-pivaloyl PGFα,
7-[3α,5α dihydroxy-2-(3a-hydroxy-5-1E-pentenyl)cyclopentyl]-5Z-heptenoic acid,
isopropyl (Z)-7-[(1R,2R,3R,5S)-3,5-dihydroxy-2-[(3R)-3-hydroxy-5-phenylpentyl]cyclopentyl]-5-heptenoate or
13,14-dihydro-15-keto-20-ethyl-PGF2α, isopropyl ester trimethylphenol-1-acetate.

32. The formulation of claim 31 wherein the topical carbonic anhydrase inhibitor is
2,3-dihydro-2-oxo-6-sulfamoyl-H-thieno-[2,3-b] [1,4]thiazine;
(2,3-dihydro-2-oxo-6-sulfamoyl-H-thieno-[2,3-b][1,4]thiazin-3-yl)acetic acid;
2,3-dihydro-2,4-dioxo-6-sulfamoyl-H-thieno-[2,3-b] [1,4]thiazine;
3-(2-hydroxyethyl)-2,3-dihydro-6-sulfamoyl-1H-thieno[2,3-b][1,4]thiazine;
3-(2-hydroxyethyl)-2,3-dihydro-4,4-dioxo-6-sulfamoyl-1H-thieno[2,3-b][1,4]thiazine;
methyl(2,3-dihydro-2,4,4-trioxo-6sulfamoyl-H-thieno-[2,3-b][1,4]thiazin-3-yl)acetate;
methyl(2,3-dihydro-2-oxo-6-sulfamoyl-H-thieno-[2,3-b] [1,4]thiazin-3-yl)acetate; or
N-isobutyl(-2,3-dihydro-2-oxo-6-sulfamoyl-1H-thieno[2,3-b] [1,4]thiazin-3-yl)acetamide;
and the prostaglandin is
isopropyl (Z)-7-[(1R,2R,3R,5S)-3,5-dihydroxy-2-[(3R)-3-hydroxy-5-phenylpentyl]cyclopentyl]-5-heptenoate, (+)-(Z)-sodium-7-[1R,2R,3R,5S)-3,5-dihydroxy-2-[(E)-1-octenyl]cyclopentyl]-5-heptenoate sesquihydrate or
13,14-dihydro-15-keto-20-ethyl-PGF2α isopropyl ester trimethylphenol-1-acetate.

33. The formulation of claim 32 wherein the carbonic anhydrase inhibitor has an aqueous solubility greater than 10 ug/mL but less than 1000 ug/mL at pH 7.4, and a Ki of 1.0 nM or lower.

34. The formulation of claim 33 which is a suspension.

35. The formulation of claim 32 which optionally contains from about 0.1% to about 2% of gellan gum or from about 0.1% to about 2% (w/w) of xanthan gum.
